(19) United States
(12) Patent Application Publication
(10) Pub. No.: US 2013/0005762 A1
(43) Pub. Date: Jan. 3, 2013

(54) COMBINATION OF DROLOXIFENE AND CLOPIDOGREL

(75) Inventor: David John Grainger, Cambridge (GB)

(73) Assignee: TCP Innovations Limited, Cambridge (GB)

(21) Appl. No.: 13/513,539

(22) PCT Filed: Dec. 1, 2010
(86) PCT No.: PCT/GB2010/002209

§ 371 Date: Sep. 18, 2012

(30) Foreign Application Priority Data
Dec. 4, 2009 (GB) 0921334.9

(51) Int. Cl. A61K 31/4365; A61P 29/00; A61P 37/06; A61P 25/28; C07D 495/04; A61P 9/10

(52) U.S. Cl. 514/301; 546/114

(57) ABSTRACT

The invention relates to the composition and use of a medicament comprising a fixed dose combination of droloxifene and clopidogrel.

The invention relates to the composition and use of a medicament comprising a fixed dose combination of droloxifene and clopidogrel.

Many prevalent diseases of middle- and old-age involve the gradual loss of the healthy tissue architecture that was assembled during embryonic and early post-natal development. For example, in coronary artery disease the concentric three-layered structure of the blood vessel wall is disrupted by the gradual development of an atherosclerotic plaque containing cholesterol, smooth muscle cells, calcium, extracellular matrix and cells of the immune system. In autoimmune conditions, the action of antibodies directed against self-antigens mediates a chronic destruction of tissue architecture. Similarly, neurodegenerative conditions such as Alzheimer's Disease result from the deposition of insoluble extracellular matrix protein aggregates and focal recruitment of activated immune cells.

More than a decade ago, we proposed that the maintenance of healthy architecture in a wide range of adult tissue s was an active process, and that cytokines in the transforming growth factor type beta (TGF-beta) superfamily were important mediators of this active maintenance (see for example Biochem Soc Trans. 1995 May; 23(2):403-6; Biol Rev Camb Philos Soc. 1995 November; 70(4):571-96). This proposition, termed the Protective Cytokine Hypothesis, was initially controversial, but has subsequently been supported by a wide variety of experimental data (see, for example, Arterioscler Thromb Vasc Biol. 2004 March; 24(3):399-404 and the references therein). For example, when mice are made partially deficient in TGF-beta (whether by heterozygous deletion of the tgfb1 gene or administration of neutralising antibodies or soluble receptors), their susceptibility to atherosclerosis is markedly increased (J Cell Sci. 2000 July; 113(13):2355-61; Arterioscler Thromb Vasc Biol. 2002 Jun. 1; 22(6):975-82; Circ Res. 2001 Nov. 9; 89(10):930-4; Blood. 2003 Dec. 1; 102(12):4052-8). Similarly, reduced levels of TGF-beta in genetically modified animals have also been shown to increase pre-disposition to cancer (for example, Nat Med. 1998 July; 4(7):802-7) and autoimmune diseases (J Autoimmun. 2000 February; 14(1):23-42).

If reduced levels of TGF-beta predisposes an individual to diseases associated with gradual loss of adult tissue architecture, such as atherosclerosis, autoimmune diseases and neurodegenerative diseases, then agents which increase TGF-beta levels should consequently be protective (see for example Nat Med. 1996 April; 2(4):381-5; Curr Alzheimer Res. 2005 April; 2(2):183-6).

Unfortunately, however, excessive levels of TGF-beta can be as damaging as reduced levels. Members of the TGF-beta family of cytokines are among the most powerful inducers of extracellular matrix formation known. As a result, if levels of TGF-beta become too high then tissue architecture becomes disrupted through exuberant production of matrix proteins such as collagen or fibronectin, which eventually disrupt the ordered relationship between the cells that compose the tissue (see for example Proc Natl Acad Sci USA. 1993 Nov. 15; 90(22):10759-63 for the effects of excessive TGF-beta on blood vessel wall architecture).

Consequently, it soon became clear that the optimal intervention for the prevention of diseases associated with a loss of adult tissue architecture would be administration of an agent or agents capable of maintaining the level of TGF-beta in the optimal range.

Direct administration of TGF-beta protein is unlikely to fulfil this criterion: like most proteins, TGF-beta shows poor pharmacokinetics (being cleared from the blood within minutes of administration (J Clin Invest. 1991 January; 87(1):39-44)) ensuring that continuous administration would be required to prevent peaks and troughs in the tissue concentration of the protein, taking the level outside of the desired optimal range.

In contrast, stimulation of the cellular production of TGF-beta exploits the natural regulatory systems that prevent (under normal circumstances) an excess activity of this fibrogenic cytokine from building up.

TGF-beta is produced as a latent precursor, which has no known biological activity. This precursor consists of a disulphide-linked dimer of the TGF-beta gene product, each monomer of which has undergone proteolytic cleavage between the mature cytokine and the LAP (or Latency-Associated Peptide). However, the dimeric LAP remains non-covalently associated with the mature cytokine, and this complex is unable to bind to conventional TGF-beta receptors. Once released into the extracellular environment (possibly associated, via covalent or non-covalent interactions, with a range of different TGF-beta binding proteins), the latent precursor is subjected to an activation step. A wide range of conditions, at least in vitro, result in a conformational change within the LAP (including application of heat, extremes of pH, chaotropic agents, proteases and specific protein:protein interactions, for example with integrins) that splits apart the non-covalent complex. The process is illustrated in FIG. 1.

This activation process is tightly regulated and serves a number of important functions: (1) it allows TGF-beta to be made by one cell type and then localised into the extracellular matrix at a distant site, where it is subsequently activated to have its effects on the nearby cells; (2) it allows a wider range of factors to dynamically control the levels of TGF-beta activity than would be possible if only gene transcription, translation and excretion were regulated; (3) it allows for feedback control to prevent dangerously high levels of TGF-beta activity building up.

One such positive feedback loop is mediated by the protease inhibitor Plasminogen Activator Inhibitor-1 (PAI-1). The levels of PAI-1 are dramatically regulated at the transcriptional level in most cells by TGF-beta activity, via the conventional TGF-beta cell surface receptors (J Biol Chem. 1991 Jan. 15; 266(2):1092-100). As a result, as TGF-beta levels rise, so do levels of PAI-1 production. PAI-1 is well known to act as an inhibitor of TGF-beta activation (J Cell Biol. 1990 August; 111(2):757-6), although the precise molecular mechanism through which the inhibition is mediated remains somewhat controversial. It is likely that PAI-1 acts either to inhibit the action of a protease involved in the intracellular cleavage between the LAP and mature cytokine during the initial production of the latent TGF-beta precursor, or else to inhibit an enzyme (again most likely a protease) which cleaves LAP to release the active cytokine (see Bioessays. 2006 June; 28(6):629-41 for a discussion of these issues).

Since PAI-1 production is stimulated by TGF-beta activity, and itself inhibits TGF-beta activation, this forms a powerful feedback loop that prevents the levels of TGF-beta activity rising too high in a particular tissue. However, since TGF-beta stimulates the production of other protease inhibitors (for example, Tissue-inhibitors of Metalloproteinases; TIMPs) it is likely that multiple parallel feedback loops exist, which together provide ample protection against excess production of the latent precursor.

Unfortunately, direct administration of active TGF-beta protein (either by pharmacologic administration, or by genetic manipulation using altered TGF-beta genes encoding a spontaneously active version of the cytokine) bypasses these regulatory processes, and allows excessive levels of TGF-beta activity to build up. In such studies, rampant tissue fibrosis is usually seen, with rapid destruction of tissue architecture.

In contrast, administration of agents that stimulate production of the latent TGF-beta precursor can increase TGF-beta activity in any tissue where the level is sub-optimal without risking excessive activity and resulting fibrogenesis. For this reason, we postulated that TGF-beta Production Stimulators would be a useful new class of therapeutic agents for the treatment of diseases associated with the loss of the adult tissue architecture, including, but not limited to, cardiovascular diseases, autoimmune diseases, and neurodegenerative diseases (see for example U.S. Pat. No. 7,084.171 issued 1 Aug. 2006; U.S. Pat. No. 6,410,587 issued 25 Jun. 2002)

One such class of TGF-beta Production Stimulators are the triphenylethylene (TPE) derivatives, such as Tamoxifen (TMX). Initially developed as estrogen receptor modulators, the TPEs as a class have diverse pharmacological activities. In addition to binding to the two estrogen receptor proteins (ER□ and ER□), various TPEs have been reported to act as inhibitors of the ATP-binding Cassette transporter proteins (Biochem Biophys Res Commun. 1997 Jun. 27; 235(3):669-74), the enzyme sterol □7,8 isomerase (J Clin Oncol. 1995 December; 13(12):2900-5) and the P-glycoprotein transporter (Biopharm Drug Dispos. 2004 October; 25(7):283-9), as well as acting as antioxidants (Biochem Soc Symp. 1995;61:209-19). In addition, however, a number of TPEs, and most particularly Tamoxifen, have been reported to stimulate the production of TGF-beta in a wide variety of cell types, both in vitro (Am J Clin Oncol. 1991; 14 Suppl 2:S15-20; Biochem J. 1993 Aug. 15; 294(1):109-12) and in vivo (J Steroid Biochem Mol Biol. 1993 December; 47(1-6):137-42; Nat Med. 1995 October; 1(10):1067-73).

It was this activity as a TGF-beta Production Stimulator which led us to claim the use of TPEs, such as Tamoxifen, for the prevention of diseases associated with the loss of normal adult tissue architecture, including cardiovascular diseases (such as coronary artery disease and restenosis), as well as autoimmune disorders and neurodegenerative disorders (for example in U.S. Pat. No. 7,084,171 and related patents).

Over the past decade, a wide variety of clinical data has been collected that support our granted claims (for example in U.S. Pat. No. 5,472,985; U.S. Pat. No. 5,595,722; U.S. Pat. No. 5,599,844; U.S. Pat. No. 5,770,609; U.S. Pat. No. 5,773,479; U.S. Pat. No. 5,847,007; U.S. Pat. No. 5,945,456; U.S. Pat. No. 6,117,911; U.S. Pat. No. 6,166,090; U.S. Pat. No. 6,197,789; U.S. Pat. No. 6,251,920; U.S. Pat. No. 6,262,079; U.S. Pat. No. 6,395,494; U.S. Pat. No. 6,410,587 and U.S. Pat. No. 7,084,171 which are each incorporated by reference herein) that TPEs, and Tamoxifen in particular, can be used to prevent these diseases associated with loss of normal adult tissue architecture, and in particular prevent death from myocardial infarction secondary to coronary artery disease. For example, Braithwaite and colleagues presented a meta-analysis of the cardiovascular outcomes of more than 27,000 women treated with Tamoxifen for the prevention of breast cancer, and found a relative risk of 0.67 for death from myocardial infarction among chronic Tamoxifen users (J Gen Intern Med. 2003 November; 18(11):937-47). This translates to a 33% reduction in risk, which, if replicated among higher risk groups such as men, would result in at least 10,000 fewer deaths due to myocardial infarction in the UK alone each year, and five times that number in the US. Similarly, Clarke and colleagues demonstrated that Tamoxifen treatment improved endothelial function, a surrogate marker of atherosclerotic disease burden (Circulation. 2001 Mar. 20; 103(11):1497-502). These results have been summarised in our recent review (Grainger & Schofield, Circulation (2005) 112:3018-24, which is incorporated by reference herein).

Unfortunately, despite such positive demonstration of efficacy in at least one disease associated with loss of normal adult tissue architecture, Tamoxifen has yet to be widely adopted for use outside of the treatment and prevention of ER-positive breast carcinoma (an application which dominantly depends on its alternative pharmacological function as an estrogen receptor modulator).

The reason for this apparent lack of enthusiasm is the burdensome side-effects which accompany the use of Tamoxifen. It is unsurprising that Tamoxifen has a range of effects (some beneficial, others less so) because of the plethora of pharmacologic and molecular interactions reported for it, as well as other members of the TPE class. Few small molecule pharmaceutical agents in use today are genuinely specific for their intended target, and side-effects frequently limit the application of otherwise highly effective medications.

There are a number of generic approaches which can be adopted to limit the impact of side-effects during drug design and development. One approach would be to design or identify entirely new compositions that retain the intended beneficial effects of the original agent, but are more specific and have less diverse molecular interactions and pharmacologic impacts. However, this approach has several major drawbacks: firstly, there is no generally successful method for identifying such compositions, and it may have been difficult, time consuming and costly to identify even the original agent with the side-effects. Secondly, some or all of the side-effects may be a direct or indirect consequence of the same molecular interaction(s) that were responsible for the target beneficial effect. In these instances it will be almost impossible to retain the profile of beneficial effects independently from the side-effects.

A second approach, which has previously been used successfully elsewhere, is to combine more than one active ingredient into a single composition, the combination having superior properties to either component administered alone, or to the same two ingredients administered to the same individual but at different times.

Two different concepts underlie the success of the combination approach: in one scenario two drugs which have similar effects but differing molecular mechanisms of action are combined, such that the two ingredients show a synergistic impact on the target factor. By using two ingredients acting synergistically it is possible to administer markedly lower doses of each ingredient in order to achieve the same beneficial effect. Provided the side-effects do not also show synergistic increases (which, provided they depend on molecular interactions which differ from the target effect, they like will not) such a composition will likely give the same beneficial effects with a reduced burden of side-effects. Indeed, even if the two agents show only additive (as opposed to synergistic) effect then a combined composition will still show reduced side-effects for the same degree of beneficial effect (although the benefit of administering them as a single composition rather than as two separate treatments will likely be less marked). There are numerous examples of such compositions, which combine two active ingredients in a single preparation. For example, Plachetka et al (U.S. Pat. No. 5,872,145 dated Feb. 16, 1999) invented a combination of a 5-HT receptor agonist with an analgesic, particularly an NSAID, for the treatment of migraine. Both active ingredients were administered at a dose below those ordinarily considered as the minimum effective dose for each agent separately, such that the combination together achieved a level of efficacy more commonly associated with administering higher doses of the single agents, each of which is accompanied by unwanted side-effects at doses above the minimum effective dose.

In the second scenario, the second active ingredient in the composition is intended to counter the side-effects of the first active ingredient, so that the combination is simultaneously effective and safe. Such compositions are less common, but patented examples have been very successful in certain applications. For example, the use of estrogen-only hormone replacement therapy leads to undesirable uterine hypertrophy, but the combination of estrogen with a progestogen leads to a combined tablet which can be used safely in women with an in tact uterus, although the unopposed estrogen is equally effective when used in women with hysterectomy (where the side-effects cannot manifest themselves). In this example, it is clearly of considerable clinical advantage to combine the two active ingredients in a single composition because the side-effects are sufficiently severe, and may even (in the case of endometrial cancer) be life-threatening, that the single combined composition precludes the possibility of the patient taking one active ingredient without the other.

TPEs such as Tamoxifen have good activity as TGF-beta Production Stimulators, but a number of side-effects have been identified which limit their broader application. Most importantly, chronic use of Tamoxifen at the most commonly used dose (20 mg/day) results in a small but significant increase in thromboembolic events, a proportion of which may be fatal. This increased pro-coagulant tendency among chronic Tamoxifen users may also underlie the increase in fatal cerebrovascular accidents (strokes) among Tamoxifen users (J Gen Intern Med. 2003 November; 18(11):937-47), some 90% of which are ischemic (as opposed to haemorrhagic in origin). These pro-coagulant side-effects are of particular concern in a cardiovascular setting where TPEs were envisioned for the prevention or treatment of coronary artery disease, since the patient may already show pro-coagulant tendencies prior to beginning treatment. Furthermore, patients at increased risk of coronary artery disease are also likely to be at increased risk of ischemic stroke. Other side effects, such as the increased risk of endometrial cancer, may also be of concern, particularly when using TPEs to treat diseases that are prevalent in women, such as autoimmune diseases (e.g. rheumatoid arthritis). More minor side-effects also exist, such as hot flushes and other consequences of the hormonal activity of the TPEs. These more minor side effects significantly affect the quality of life of the patient, and while they would not necessarily preclude the use of these agents for the treatment of severe or life-threatening conditions (including the diseases associated with loss of the normal adult tissue architecture, such as cardiovascular diseases, autoimmune disorders and neurodegeneration), such side-effects cause problems with patient compliance which in turn threatens the effectiveness of such medications even for the treatment of more severe disease.

We have previously described a series of compositions useful as TGF-beta Production Stimulators for the prevention or treatment of diseases associated with the loss of normal adult tissue architecture (including cardiovascular diseases, autoimmune diseases, and neurodegenerative conditions) which reduce or avoid the side-effects which otherwise limit the application of previously described TGF-beta Production Stimulators in these broad indications (WO 2008/099144; GB 2446641). These compositions comprised at least two active ingredients (as well as any excipient or carrier), where at least one of the active ingredients was a TGF-beta Production Stimulator, and another active ingredient was able to reduce the side-effects associated with the administration of the first active ingredient.

Our original disclosures (WO 2008/099144; GB 2446641) described a large number of possible combinations, with variation in both the selected TGF-beta Production Stimulator and the second (and additional) active ingredients intended to mitigate the side-effects of administering the selected TGF-beta Production Stimulator. A number of different examples were provided to demonstrate the benefit of the claimed compositions. However, the specification and claims provided little guidance on the selection of the most useful combinations among the many that were disclosed.

It is clear that TPEs related to Tamoxifen, having the structure (I), or more preferably (II) as listed in our original disclosure are preferred TGF-beta Production Stimulators for use according to the prior invention. However, a sufficiently large number of chemical compounds fall under even the narrower generic formula (II) that it would be impractical to test them all in order to discover which (if any) have superior properties over the general class.

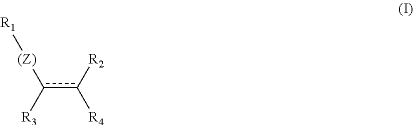

wherein

R₁ is (C1-C6)alkyl, or aryl, optionally substituted by 1, 2 or 3 V;

R₂ is phenyl, optionally substituted by 1, 2 or 3 V; or R₂ is (C1-C12)alkyl, halo(C1-C12)alkyl, (C3-C6)cycloalkyl, (C1-C6)alkylcyclo(C3-C6)alkyl, (C5-C6)cycloalkenyl, or (C1-C6)alkyl(C5-C6)cycloalkenyl;

R₃ is hydrogen or phenyl, optionally substituted at the 2-position with Rⱼ, and additionally optionally substituted by 1,2 or 3 V;

R₄ is hydrogen, nitro, halo, aryl, heteroaryl, aryl(C1-C3)alkyl, heteroaryl(C1-C3)alkyl, halo(C1 -C1 2)alkyl, cyano(C1-C12)alkyl, (Cl -C4)alkoxycarbonyl(C1-C12)alkyl, (C1-C12)alkyl, (C3-C6)cycloalkyl, (C1-C6)alkylcyclo(C3-C6)alkyl, (C5-C6)cycloalkenyl, or (C1-C6)alkyl(C5-C6)cycloalkenyl, wherein any aryl or heteroaryl may optionally be substituted by 1,2 or 3 V; or

R₅ and Rⱼ together are —CH₂—CH₂—, —S—, —O—(NH)—, —N[C1-C6)alkyl]-, —OCH₂—, —O—C[(C1-C6)alkyl]₂— or —CH═CH—;

- - - is a single bond or is —C(B)(D)- wherein B and D are each independently hydrogen, (C1-C6)alkyl or halo;

V is OPO₃H₂, (C1-C6)alkyl, (C1-C6)alkoxy, mercapto, (C1-C4)alkylthio, halo, trifluoromethyl, perntafluoroethyl, nitro, N(Rₙ)(Rₒ), cyano, trifluoromethoxy, pentafluroethoxy, benzoyl, hydroxy, alkyl, benzyl, —OSO₂(CH₂)₀₋₄CH₃, U(CH₂)₁₋₄COORp, —(CH₂)₀₋₄COORₚ, —U(CH₂)₂₋₄ORₚ, —(CH₂)₀₋₄ORₚ, —U(CH₂)₁₋₄C(═O)Rₖ, —(CH₂)₀₋₄C(═O)Rₖ, —U(CH₂)₁₋₄Rₖ, —(CH₂)₀₋₄Rₖ, or —U(CH₂)₂₋₄OC(═O)Rₚ; wherein U is O, N(Rₘ), or S;

Z is —(CH₂)₁₋₃—, O, —OCH₂—, —CH₂O—, —C(═O)O—, N(R_{q})—, C═O, or a covalent bond;

Rₖ is amino, optionally substituted with one or two (C1-C6)alkyl; or an N-heterocyclic ring optionally containing 1 or 2 additional N(R_{z}), S or nonperoxide O, wherein R_{z} is H, (C1-C6)alkyl, phenyl or benzyl;

Rₙ and Rₒ are independently hydrogen, (C1-C6 alkyl), phenyl, benzyl, or (C1-C6)alkanoyl; or Rₙ and Rₒ together with the nitrogen to which they are attached are a 3,4,5 or 6-membered heterocyclic ring;

Rₚ is H or (C1-C6)alkyl; and

Rₘ and R_{q} are independently hydrogen, (C1-C6)alkyl, phenyl, benzyl or (C1-C6)alkanoyl;

or the compound is 1-(4-[2-(diethylamino)ethoxylphenyl)-2-(4-methoxyphenyl)-1-phenylethan-1-ol (MER25);

or a pharmaceutically acceptable salt thereof.

The preferred compounds of general formula (I) were compounds with the triphenylethylene structure of formula (II):

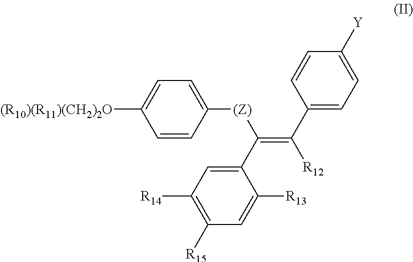

wherein

Z is C═O or a covalent bond;

Y is H or O(C1-C4 alkyl);

R₁₀ and R₁₁ are individually (C1-C4)alkyl or together with the N to which they are bound form a saturated heterocyclic group;

R₁₂ is ethyl or chloroethyl;

R₁₃ is H, or together with R₁₂ is —CH₂—CH₂— or —S—;

R₁₄ and R₁₅ are independently selected among H, I, O(C1-C4)alkyl;

or a pharmaceutically acceptable salt thereof.

It is also clear that an anti-platelet agent such as aspirin, an aspirinate or a thiphene of formula (III) is a preferred second agent in the composition according to the earlier invention. However, a sufficiently large number of chemical compounds full under these generic formulae that it would be impractical to test them all to discover which (if any) have superior properties over the general class.

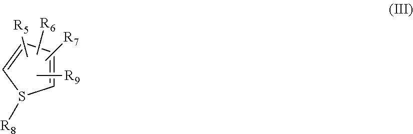

where R₅ is hydrogen, halo, nitro, cyano, hydroxy, CF₃, —NR_{c}R_{d}, —C(═O)ORₑ, —OC(═O)ORₑ, —C(═N)ORₑ, (C1-C6)alkyl or (C1-C6)alkoxy;

R₆ is hydrogen or —XRₐ;

R₇ is —C(═O)YR_{b};

R₈ is (═O)ₙ or R₅ is (C1-C6)alkyl, (C1-C6)alkanoyl or (C1-C6)alkanoyloxy and forms a sulfonium salt with the thiophene sulphur, wherein the associated counter ion is a pharmaceutically acceptable anion ;

R₉ is hydrogen, —C(═O)ORₕ or —C(═O )SRₕ;

n=0,1 or 2;

X is oxygen or sulphur;

Y is oxygen or sulphur ;

Rₐ is (C1-C6)alkanoyl ;

R_{b} is hydrogen or (C1-C3) alkyl ;

R_{c} and R_{d} are each independently hydrogen, (C1-C4)alkyl, phenyl, C(═O)OH, C(═O)O(C1-C4)alkyl, CH₂C(═O)OH, CH₂C(═O)O(C1-C4)alkyl, or (C1-C4)alkoxy; or R_{c} and R_{d} together with the nitrogen to which they are attached are a 3,4,5 or 6 membered heterocyclic ring ; and

Rₑ-Rᵢ are independantly hydrogen or (C1-C6)alkyl ; or a pharmaceutically acceptable salt thereof;

provided that R₆ and R₇ are on adjacent positions of the ring to which they are attached, or are on the 2- and 5-positions of the ring ; and further provided that when R₆ is hydrogen R₇ is on the 2- or 5-position of the ring to which it is attached and R₄ is (C1-C4)alkanoyloxy.

It is evident that where a composition is claimed that is a combination of two agents freely selected from two different classes that the number of composition this covered is the product of the number of members of each component class. Thus, where only 100 agents were members of the first class and only 100 different agents were members of the second class, 10,000 different combinations could be formed according to the invention previously disclosed. With respect to WO 2008/099144 and GB 2446641, it is evident that many more than 100 agents are members of the disclosed class of TGF-beta Production Stimulators. Furthermore, many more than 100 agents are members of the class of anti-platelet agents (including aspinates and compounds of formula (III)). As a result very many more than 10,000 combinations of these classes are disclosed and claimed therein.

From this very large class of generically disclosed combinations, a smaller number (based primarily on the amount of data available for them, rather than on any disclosed superior properties of those combinations) of preferred compositions were disclosed:

In particular, preferred compositions according to the prior disclosure were to be selected from the following list:
Tamoxifen and aspirin, droloxifene and aspirin or toremifene and aspirin ;
Tamoxifen and clopidogrel, droloxifene and clopidogrel or toremifene and clopidogrel;
Tamoxifen and aspirin and clopidogrel;
Tamoxifen and atorvastatin or Tamoxifen and simvastatin;
Tamoxifen aspirinate, droloxifene aspirinate or toremifene aspirinate;
Tamoxifen aspirinate and clopidogrel or Tamoxifen aspirinate and atorvastatin
Tamoxifen and naproxen or Tamoxifen aspirinate and naproxen;
Tamoxifen and galantamine or Tamoxifen aspirinate and galantamine and (except where specific salts are already specified) any pharmaceutically acceptable salts thereof.

Of these eighteen combinations, twelve contain Tamoxifen as the preferred TGF-beta Production Stimulator. Furthermore, the prior disclosure teaches that a combination of Tamoxifen and Clopidogrel is the most preferred embodiment of the previous invention. Furthermore all of the examples provided select Tamoxifen as the TGF-beta Production Stimulator.

Dicker et al. (U.S. Patent Application Publication No. 2004/0180812) also disclose the use of both a triphenylethylene and an anti-platelet agent for the treatment of proliferative disorders such as cancer. While Dicker et al. does not disclose a composition combining a triphenylethylene with an anti-platelet agent (and instead proposes a treatment regimen where pre-treatment with the anti-platelet agent improves the access of the anti-proliferative triphenylethylene to the locality of the tumour, where they expect it to be most active), nevertheless when selecting among appropriate agents to include in their lists of agents useful according to their invention, they selected Tamoxifen and Clopidogrel for inclusion.

Prior to the present invention, therefore, there is no reason to suppose that any one out of a very large number of combinations of agents previously disclosed would be particularly superior to the others based on the data presented. The available teaching, both from our earlier work and from Dicker et al who were looking to solve an altogether different problem, strongly suggests that combinations comprising Tamoxifen as the chosen triphenylethyene together with another agent would be preferable, and in particular that the most preferable combination would be Tamoxifen and Clopidogrel.

Here we demonstrate that, surprisingly, one particular combination of a TGF-beta Production Stimulator and an anti-platelet agent is markedly and unexpectedly superior to the general class of combinations we have previously disclosed. In particular, the combination of the TPE droloxifene and the anti-platelet agent clopidogrel is dramatically superior to the combination of Tamoxifen and clopidogrel, which, according to the prior teaching, one might have supposed to be the most preferable such combination.

More specifically, the present invention provides the composition and use of a therapeutic agent, comprising two active ingredients, where the first agent is droloxifene (IV), either as the free base or as a pharmaceutically acceptable salt form, and the second agent is clopidogrel (V), either as the free base or as a pharmaceutically acceptable salt form.

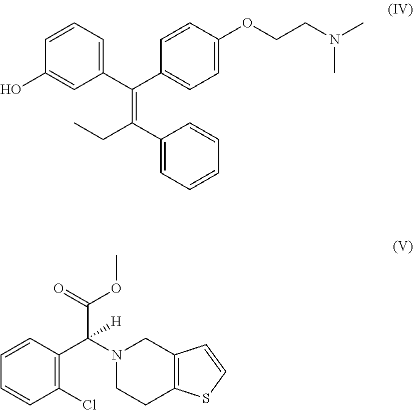 [SMILES: CC/C(=C(/c1ccc(OCCN(C)C)cc1)c1cccc(O)c1)c1ccccc1 ; COC(=O)[C@H](c1ccccc1Cl)N1CCc2sccc2C1]

Importantly, the composition of the invention must be administered to the patient as a mixture. The principal advantage of the composition of the invention over the separate administration of the two active ingredients is safety. The side effects of triphenylethylenes, such as droloxifene, can be severe and even lethal in certain circumstances. As a result, it represents an unnecessary risk to allow the administration of the two agents separately, when the possibility exists that the patients may (accidentally or deliberately) continue to have administered one of the active ingredients and not the other active ingredient. In such circumstances, the patient may suffer considerable harm: the triphenylethylenes increase the risk of thromboembolism (and hence stroke), while the anti-platelet agent mitigates this risk.

In other words, the provision of these two pharmaceutical agents as a single medicament (tablet, capsule, gel or other dosage form) offers considerable advantages over the separate administration of the two pharmaceutical agents, when the desirable effect of the two components together is different from the effects of either agent when administered separately. Although the same effect might, in principle, be achievable by administering the compounds at the same time but as separate medicaments (tablets, capsules or gels, for example), nevertheless the risk of achieving a different (and less desirable) effect similar to either compound administered alone is greater than when the two are administered as a single medicament. Where the difference in effect profile between the combination of the two pharmaceutical agents and either one administered alone is significant (such as in the case of potentially lethal side-effects) then such an increased risk becomes unacceptable.

Furthermore, as demonstrated herein, the metabolic interactions between the two agents will likely result in unpredictable levels of the active metabolite of clopidogrel if the timing of ingestion of the two agents is not simultaneous. On this basis, unexpected (and potentially deleterious) outcomes are increasingly likely as the possibility of the patient taking the two components of the combined medicament at separate times increases. The presence of the two components in the same dosage form ensures simultaneous co-administration and therefore results in a more predictable exposure profile to the active ingredients (and their metabolites) and therefore a more predictable, and desirable, therapeutic outcome.

The invention also provides pharmaceutical compositions comprising at two active ingredients as a mixture, including droloxifene (IV) or a pharmaceutically acceptable salt thereof, together with clopidogrel (V) or a pharmaceutically acceptable salt thereof, and at least one pharmaceutically acceptable excipient and/or carrier. For the purposes of this specification, the term ‘mixture’ may optionally include the chemical combination of the two agents (for example, as an ester, or an amide or any similar covalent chemical linkage which allows both components to retain their full pharmaceutical activity). For example, the ester formed by transesterification of the methyl ester in clopidogrel with the hydroxyl group of droloxifene for form a single molecule (VI) would represent a ‘mixture’ of droloxifene and clopidogrel according to the present invention, and is therefore claimed.

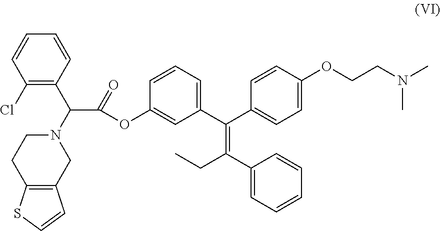 [SMILES: CC/C(=C(/c1ccc(OCCN(C)C)cc1)c1cccc(OC(=O)C(c2ccccc2Cl)N2CCc3sccc3C2)c1)c1ccccc1]

It will be evident that compound (VI) can exist in more than one optically-active form. As a result, the present disclosure relates to the either enantiomer in isolation, as well as to mixtures of the enantiomers including a racemic mixture of both enantiomers in equal proportions. Compound (VI) may also be readily prepared in the form of a salt, with a wide range of counterions known in the art, and such salt forms are encompassed by the current disclosure.

However, since clopidogrel is known to only possess anti-platelet activity in one stereochemical arrangement, the optical isomer of (VI) which would yield as a metabolite following cleavage of the ester bond a compound with the spatial arrangement of atoms as in clopidogrel is preferred. This preferred optical isomer of (VI) is illustrated as (VI′). For the avoidance of doubt, this preferred isomer has the trans (or (E)-) configuration around the double bond, as illustrated.

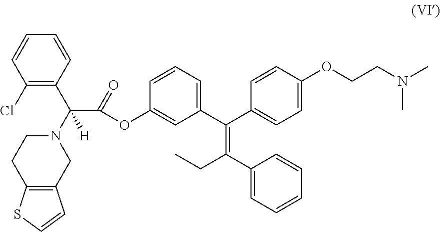 [SMILES: CC/C(=C(/c1ccc(OCCN(C)C)cc1)c1cccc(OC(=O)[C@H](c2ccccc2Cl)N2CCc3sccc3C2)c1)c1ccccc1]

By pharmaceutically acceptable salt is meant in particular the addition salts of inorganic acids such as hydrochloride, hydrobromide, hydroiodide, sulphate, phosphate, diphosphate and nitrate or of organic acids such as acetate, maleate, fumarate, tartrate, succinate, citrate, lactate, methanesulphonate, p-toluenesulphonate, palmoate and stearate. Also within the scope of the present invention, when they can be used, are the salts formed from bases such as sodium or potassium hydroxide. For other examples of pharmaceutically acceptable salts, reference can be made to “Salt selection for basic drugs”, Int. J. Pharm. (1986), 33, 201-217. Preferably the droloxifene component will be present as the citrate salt. Preferably the clopidogrel component will be present as the bisulphate (or hydrogen sulphate) salt.

Typically, the composition will include an amount of droloxifene in the range 1 mg to 200 mg, more typically 10 mg to 100 mg. Typically, the composition will include an amount of clopidogrel in the range 10 mg to 500 mg, more typically 50 mg to 200 mg. Typically, the ratio of clopidogrel to droloxifene will be in the range 10:1 to 1:10, more typically in the range 10:1 to 1:1. A preferred composition according to the invention includes droloxifene (as the citrate salt) at a dose 20-80 mg together with clopidogrel (as the bisulphate salt) at a dose of 50-150 mg.

A preferred composition according to the invention consists of 20 mg droloxifene citrate combined with 75 mg of clopidogrel, the said composition in tablet form (with appropriate pharmaceutical carriers or excipients). Preferably tablets of such composition would be administered to the patient on a single occasion each day.

The pharmaceutical composition can be in the form of a solid, for example powders, granules, tablets, gelatin capsules, liposomes or suppositories. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax. Other appropriate pharmaceutically acceptable excipients and/or carriers will be known to those skilled in the art.

The pharmaceutical composition according to the invention can also be presented in liquid form, for example, solutions, emulsions, suspensions or syrups. Appropriate liquid supports can be, for example, water, organic solvents such as glycerol or glycols, as well as their mixtures, in varying proportions, in water.

The invention includes compounds, compositions and uses thereof as defined, wherein the compound is in hydrated or solvated form.

According to this invention, disorders intended to be prevented or treated by the compositions of the invention, or the pharmaceutically acceptable salts thereof or pharmaceutical compositions or medicaments containing them as active ingredients include notably:
autoimmune diseases, for example such as multiple sclerosis, rheumatoid arthritis, Crohn's disease, Grave's disease, mysethenia gravis, lupus erythromatosis, scleroderma, Sjorgren's syndrome, autoimmune type I diabetes;
vascular disorders including stroke, coronary artery diseases, myocardial infarction, unstable angina pectoris, atherosclerosis or vasculitis, e. g., Behçet's syndrome, giant cell arteritis, polymyalgia rheumatica, Wegener's granulomatosis, Churg-Strauss syndrome vasculitis, Henoch-Schönlein purpura and Kawasaki disease;
asthma, allergic rhinitis or chronic occlusive pulmonary disease (COPD);
osteoporosis (low bone mineral density);
tumor growth;
organ transplant rejection and/or delayed graft or organ function, e.g. in renal transplant patients;
psoriasis;
allergies;
Alzheimer's disease, and other idiopathic dementias resulting from neurodengeneration;
Parkinson's disease;
Huntington's disease;
Traumatic brain injury (such as head injuries resulting from a motor vehicle accident), as well as the chronic sequelae (such as impaired memory) resulting from such acute traumatic injuries

Where legally permissible, the invention also provides a method of treatment, amelioration or prophylaxis of the symptoms of a disease involving the loss of normal adult tissue architecture by the administration to a patient of a therapeutically effective amount of a composition or medicament as claimed herein.

Administration of a medicament according to the invention can be carried out by topical, oral, parenteral route, by intramuscular injection, etc.

Preferably, the diseases ameliorated, treated or prevented by the administration of the compositions of the invention are selected from the following list:
Cardiovascular diseases, including atherosclerosis, and the clinical sequellae of atherosclerosis, such as myocardial infarction, angina pectoris, unstable angina, stroke, transient ischemic attack and peripheral occlusive artery disease
Autoimmune diseases, including rheumatoid arthritis and multiple sclerosis
Neurodegenerative diseases, including Alzheimer's Disease and Parkinson's Disease
Tumour growth

The compositions of the invention are readily manufactured using methods that are well known in the art. In particular, the individual active pharmaceutical ingredients may be synthesised by methods well known in the art, and both are commercially available isolated chemical compounds. Except where the two active ingredients are chemically combined, the two active pharmaceutical ingredients that compose the composition of the invention are then mixed together, preferably as a finely divided powder so that a homogenous mixture is achieved, then added to appropriate pharmaceutical carriers and/or excipients using techniques well known in the art. The mixture, together with any carriers and excipients, is then prepared in a form suitable for administration to a human, for example as a tablet, capsule, liquid suspension or suppository, using methods well established in the art.

Where the composition of the invention includes two or more active pharmaceutical ingredients which are chemically combined, for example as in the ester (VI), then the combination is prepared using methods well known in the art. For example, to prepare ester (VI) 3′-Carboxymethylene-4′-mercapto-piperidin-1-yl)-(2″-chlorophenyl)acetic acid is converted into an acid chloride (using thionyl chloride or other method) or an active ester (using HATU or other coupling agent) and then treated with droloxifene and 4-dimethylaminopyridine to form an ester, according to standard methods. Alternatively an equimolar mixture of 3′-carboxymethylene-4′-mercapto-piperidin-1-yl)-(2″-chlorophenyl)acetic acid and droloxifene could be treated with dimethylaminopyridine and a carbodiimide coupling agent according to standard methods Other methods of transesterification are well known in the art, and can be similarly be used to prepare ester (VI),

The following examples are presented in order to illustrate the above procedures and should in no way be considered to limit the scope of the invention.

### EXAMPLE 1

### Droloxifene is a Comparable TGF-beta Production Stimulator to Other Triphenylethylenes, Including Tamoxifen

A wide range of triphemylethylenes have been shown to be TGF-beta Production Stimulators, and as a result the general class have been claimed for this purpose; see for example U.S. Pat. No. 6,262,079 and U.S. Pat. No. 6,410,587). However, there has been no published comparison of the potency and power of various triphenylethylenes as TGF-beta Production Stimulators. Furthermore, it is known in the art that different triphenylethylenes have different effects on the isoforms of TGF-beta: Tamoxifen, for example, stimulates both TGF-beta1 and to a lesser extent TGF-beta3, while Raloxifene stimulates predominantly TGF-beta3 and has little or no effect on TGF-beta1. This difference in effects on these isoforms has been postulated to underlie the rather less impressive effects of Raloxifene for the prevention of coronary heart disease than for Tamoxifen (Grainger & Schofield (2005) Circulation 112:3018-24).

In order compare Droloxifene with other triphenylethylenes, we have exploited a functional assay for TGF-beta1 activity. Alternative approaches are less suitable for various reasons: it is not possible to directly measure TGF-beta protein production by cultured cells in response to triphenylethylenes because the TGF-beta that is produced remains associated with the cells or the extracellular matrix and is therefore not available for assay (for example, by ELISA) in the conditioned medium from the cultured cells. Measuring the level of messenger RNA, for example by quantitative PCR, provides an indication of the likelihood that an agent is a TGF-beta Production Stimulator, but it does not provide definitive evidence, nor allow a comparison of potency and power, because post-transcriptional processes play an important role in regulating TGF-beta activity and any effect of the test agents on these processes (such as translation efficiency, glycosylation, signal peptide removal, maturation, availability and activation) are not captured by measurement of the mRNA level. By contrast, a functional assay such as measuring the rate of proliferation of smooth muscle cell cultures, which is potently inhibited by TGF-beta, provides a measure of the aggregate effect of the test agent on all of these processes. The specificity of such an assay is achieved by comparing the rate of proliferation of the smooth muscle cells in the presence or absence of a validated, highly specific neutralising antibody against TGF-beta (or TGF-beta1, as required). This method has been used extensively in the prior art for the demonstration of TGF-beta Production Stimulator activity of various compounds (see, for example, Kirschenlohr et al. (1995) Cardiovasc. Res. 29:848-55 or U.S. Pat. No. 6,410,587).

Methods

We selected Wistar rat aortic vascular smooth muscle cells as the target cell type for this functional assay, because these cells have previously been shown to increase TGF-beta production in response to Tamoxifen (see for example Grainger et al (1993) Biochem. J. 294(1):109-12 for in vitro evidence and Grainger et at (1998) J. Cell Sci. 111(19):2977-88 for in vivo evidence), and to respond by slowing proliferation in a manner reversible by neutralising anti-TGF-beta1 antibodies. The cells were prepared by enzyme dispersion of isolated rat aortae exactly as described previously (Grainger et al (1993) Biochem. J. 294(1):109-12 and the references therein). The cells were cultured (37° C.; 5% CO₂) in DMEM+10% foetal calf serum (FCS), and subcultured every 4 days at 1:2 dilution, using 0.02% trypsin/EDTA (Gibco). Experiments were performed between the sixth and twentieth subculture.

For the experiments, the cells were subcultured into 12-well cluster plates, and allowed to grow for 24 hrs. At this time (designated ‘0 hours’), the test agents were added to the cells, in 10% ethanol vehicle, such that the concentration of vehicle in the culture medium did not exceed 0.1%. The cells were then incubated for 72 hrs. All treatment conditions were established in triplicate. One triplicate of cells was counted (see below) at Oh. All other wells were counted at 72 hrs.

The number of cells in each well was determined as follows: the well was washed three times with serum-free DMEM, aspirated and then incubated with 100 μl of trypsin/EDTA solution (Gibco) for 2 minutes at 37° C. Complete release of the cell monolayer was confirmed microscopically. The cell density in portion of this suspension was determined by counting using an Improved Neubauer hameocytometer, ensuring the suspension was uniform by gentle pipetting, prior to sampling. The cell density was converted to the absolute number of cells present in the well by multiplying the number of cells per μl by the total volume of suspension (100 μl). For each condition, the mean number of cells at the end of the experiment was calculated by meaning the number of cells in each well of triplicate.

The extent of proliferation of the cell culture under each condition was determined by subtracting the mean number of cells at time Oh from the mean number of cells under that condition at time 72 h. The amount of TGF-beta1 activity under each condition was determined by subtracting the inhibition of growth achieved in the presence of neutralising anti-TGF-beta1 antibody (AB-101-NA; R&D Systems) from the inhibition of growth achieved in the absence of antibody.

In separate control experiments, the amount of neutralising antibody used (10 μh/ml) was shown to fully inhibit the effects of 100 ng/ml recombinant active TGF-beta1 (R&D Systems). A control antibody (non-immune chicken IgY; Sigma) was shown to have no effect on cell growth in the presence or absence of TGF-beta1.

This method is similar to that described elsewhere to determine the effects TGF-beta Production Stimulators (for example Kirschenlohr et al. (1995) Cardiovasc. Res. 29:848-55; Grainger et al (1993) Biochem. J. 294(1):109-12; Grainger et al (1993) Cardiovasc. Res. 27(12):2238-47; US 6,410,587).

Results

Tamoxifen increased TGF-beta activity in a dose-dependent fashion, with a half-maximal effect around 7 μM (FIG. 2; Table 1), consistent with previously published data (Grainger et al (1993) Biochem. J. 294(1):109-12). We note that the apparent ED50 can vary, most likely depending on the batch of fetal calf serum used in the experiment, and that again depending on the batches of serum, tamoxifen causes cell toxicity at concentrations ranging from 10 μM to 66 μM. Nevertheless, it is possible to compare the effects of different analogs of tamoxifen side-by-side in the same experiment, and the differences between the analogs cannot therefore be due to variations in the precise conditions of the experiment, such as the particular batch of serum used (which is consequently the same for all analogs tested). There was no difference in the value obtained for tamoxifen citrate and tamoxifen free base.

TABLE 1
ED50 for the TGF-beta Production Stimulator activity of various
structural analogs of Tamoxifen. The ED50 was determined from
the dose-response curves shown in FIG. 2. The degree of TGF-beta
Production Stimulator activity was calculated as the suppression of
the proliferation of rat aortic smooth muscle cells in culture that was
reversible by the presence of neutralising antibodies to TGF-beta1
(see methods above).

| Analog | ED50 (μM) |
|---|---|
| Tamoxifen | 7 |
| Droloxifene | 3 |
| Toremifene | 10 |
| Idoxifene | 5 |
| Raloxifene | >100 |

The effect of various structural analogs of Tamoxifen is shown in FIG. 2 and Table 1. With the exception of raloxifene, which was substantially less active, there was essentially no difference in either the potency or the maximum effect achievable with the other analogs of Tamoxifen. Note that Raloxifene does not have measurable activity as Production Stimulator for TGF-beta1 (measured here) but stimulates the production of TGF-beta3.

Conclusions

These experiments demonstrate that Droloxifene has comparable activity as a TGF-beta Production Stimulator to Tamoxifen, and several other commonly studied structural analogs of Tamoxifen.

### EXAMPLE 2

### Effect of Co-Administration of Triphenvlethylenes on Clopidogrel Metabolism In Vivo

Clopidogrel, like other members of the thiophene class of P2Y12 antagonists, does not exhibit anti-platelet activity directly. Instead, the molecule is activated in vivo through the action of cytochrome P450 isoenzymes, predominantly 2C9 and 2C19 (Ayalasomayajula et al. (2007) J Clin Pharmacol. 47(5):613-9; Dansette et al (2009) Chem Res Toxicol. 22(2):369-73.). Following oxidation, the thiphene ring opens to yield a reactive that covalently binds to, and inactivates, the platelet P2Y12 receptor, leading to desensitisation. Clopidogrel activation via this mechanism is particularly sensitive to the levels of cytochrome P450 activity because a competing ester hydrolysis inactivates the molecule. As a result, if the generation of the active metabolite is slowed to a degree, activity falls off much more rapidly due to ester hydrolysis.

As a result, factor which affect cytochrome P450 activity, such as genetic polymorphisms and co-administration of other xenobiotics, markedly affect clopidogrel metabolism, and its subsequent biological efficacy. For example, particular “low metabolizer” polymorphisms in 2C19 have been identified (Mega et al (2009) New Engl. J. Med. 360: 354-362; Simon et al. (2009) New Engl. J. Med. 360: 363-375; Collet et al (2009). Lancet 373:309), resulting in peak levels of the active metabolite only one third of those found in individuals with the more common wild-type alleles. In contrast, co-administration of cytochrome P450 isoenzyme inducers, such as grapefruit juice can as much as double the peak concentration of the active metabolite of clopidogrel.

Since many drugs have complex effects of the cytochrome P450 system, inhibiting some isoenzymes but also inducing others, it is difficult, if not impossible, to predict the overall impact of administering a cytochrome P450 isoenzyme modulating agent on the pharmacokinetics of the key active metabolite of clopidogrel. Furthermore, if two such drugs do interact in this way, then the impact can vary with time, since on initial administration the direct inhibition of cytochrome P450 activity may dominate but with time, after repeated dosing, the induction of the same, or different, cytochrome P450 isoenzymes may result in an overall rise in relevant P450 activity.

Treiphenylethylenes, including Tamoxifen, are known to be both substrates and inhibitors of Cytochrome P450 isoenzymes (predominantly 3A4), and to induce liver expression of a range of other isozymes (see Desta et at (2004) J Pharmacol Exp Ther. 310(3):1062-75 for an overview). As a result, there will very likely be an interaction, such that co-administration of a triphenylethylene will affect the pharmacokinetics of the active metabolite of clopidogrel in some unpredictable way, resulting an undesirable modulation of its biological and therapeutic properties. The risk of such an interaction is sufficiently high that the label of Plavix™ (clopidogrel bisulphate tablets from Bristol Myers Sqibb) approved by regulatory bodies such as the EMEA in Europe and the FDA in the USA carry a warning of such interaction. Despite the theoretical risk underpinning this label advice, the actual nature of the interaction between these two agents has not been extensively studied, and is difficult to predict.

The nature of any interaction between structurally distinct triphenylethylenes and clopidogrel metabolism will also vary in subtle, but complex and unpredictable, ways depending on the particular cytochrome P450 isoenzyme inhibition and induction profiles of the particular molecule, which can only be determined by experimental investigation.

Methods

Adult male Sprague-Dawley rats are dosed with various triphenylethylenes at different doses from 0.1 mg/kg to 10 mg/kg (spanning the likely doses such compounds would be used in man), or with vehicle only, and simultaneously dosed with 10 mg/kg clopidogrel, both by oral gavage. Blood samples are drawn through an in-dwelling jugular catheter at 0.5, 1, 2, 4, 8 and 24 hrs post-dose, and serum is prepared by conventional methods well known in the art.

The level of CLP*, the active metabolite of clopidogrel is then determined in each blood sample by LC-MS by collecting the blood samples in tubes containing the thiol alkylating agent iodeoacetamide. Iodoacetamide reacts with CLP* to generate a stable product which can be detected by LC-MS using standard methodology well known in the art. An authentic sample of the acetamide adduct of CLP* is used to quantify the level of CLP*-acetamide in the sample. The results from this assay correlate with the bioassay for antiplatelet activity which has been routinely used to estimate levels of CLP* (see for example Pereillo et al (2002) Drug Met. Disposition 30:1288-95), and it is therefore assumed that the capture of CLP* by iodoacetamide either proceeds quantitatively to completion or else is zero-order with respect to CLP* concentration under the conditions of the reaction. The concentration of CLP*-acetamide adduct can therefore be assumed to be proportional to the concentration of CLP*. The pharmacokinetics of CLP*-acetamide are then compared under the different conditions, using standard pharmacokinetics models implemented with WinNonLin.

Results

Compared to vehicle control, simultaneous administration of tamoxifen decreased the Cmax concentration of CLP*-acetamide in a dose-dependent fashion maximal at an 88% reduction at 10 mg/kg Tamoxifen. Such a severe suppression of clopidogrel metabolism will clearly have a significant and undesirable impact on the biological and therapeutic impact of the clopidogrel. This provides clear experimental evidence for the warning on the label of Plavix™, which was based on theoretical considerations.

Other analogs of Tamoxifen also markedly inhibited clopidogrel metabolism (Table 2), although none has as large an effect as Tamoxifen itself. Of the five analogs tested, only Droloxifene had no effect on clopidogrel metabolism.

TABLE 2
Effect of various structural analogs of Tamoxifen on the metabolism
of clopidogrel. Cmax for CLP*-acetamide, a marker for the active
metabolite of clopidogrel, is shown (mean of 3 animals). The
percentage inhibition compared to vehicle control is calculated, and
the statistical significance of the effect of co-dministration of the
triphenylethylene determined by application of Student's unpaired
t-test (two tailed, assuming equal variances). P values less than 0.05
were taken to indicate a significant effect of the triphenylethylene on
clopidogrel metabolism.

| Analog | Cmax (ng/ml) | % inhibition | P value |
|---|---|---|---|
| Vehicle | 5.3 ± 0.9 | 0 | n/a |
| Tamoxifen | 3.8 ± 0.7 | 28 | 0.18 |
| 0.1 mg/kg | | | |
| Tamoxifen | 2.3 ± 0.3 | 57 | 0.02 |
| 1 mg/kg | | | |
| Tamoxifen | 0.6 ± 0.1 | 88 | 0.003 |
| 10 mg/kg | | | |
| Droloxifene | 5.3 ± 0.5 | 0 | 1.00 |
| 0.1 mg/kg | | | |
| Droloxifene | 6.3 ± 1.0 | −19 | 0.41 |
| 1 mg/kg | | | |
| Droloxifene | 5.7 ± 0.4 | −7 | 0.66 |
| 10 mg/kg | | | |
| Toremifene | 4.7 ± 0.2 | 11 | 0.49 |
| 0.1 mg/kg | | | |
| Toremifene | 2.7 ± 0.1 | 48 | 0.02 |
| 1 mg/kg | | | |
| Toremifene | 1.2 ± 0.6 | 77 | 0.009 |
| 10 mg/kg | | | |
| Idoxifene | 5.2 ± 1.2 | 2 | 0.94 |
| 0.1 mg/kg | | | |
| Idoxifene | 5.0 ± 0.6 | 6 | 0.74 |
| 1 mg/kg | | | |
| Idoxifene | 2.7 ± 0.4 | 48 | 0.03 |
| 10 mg/kg | | | |
| Raloxifene | 6.3 ± 0.7 | −18 | 0.34 |
| 0.1 mg/kg | | | |
| Raloxifene | 4.7 ± 0.7 | 11 | 0.57 |
| 1 mg/kg | | | |
| Raloxifene | 2.5 ± 0.3 | 53 | 0.02 |
| 10 mg/kg | | | |

Conclusions

These experiments conclusively demonstrate that almost all triphenylethylenes affect the metabolism of clopidogrel to differing extents. Surprisingly, droloxifene has no effect on the pharmacokinetics of clopidogrel and is therefore unexpectedly superior for use in combination with clopidogrel than any of the other tamoxifen analogs tested. Indeed, with the exception of raloxifene (which cannot be used as a TGF-beta1 Production Stimulator because it does not stimulate the production of TGF-beta1—see Example 1), none of the triphenylethylene compounds tested, other than droloxifene, would be at all useful when administered in combination with clopidogrel. Droloxifene is, therefore, not only quantitatively, but qualitatively superior to all the other triphenylethylene compounds tested here.

Definitions

The term “about” refers to an interval around the considered value. As used in this patent application, “about X” means an interval from X minus 10% of X to X plus 10% of X, and preferably an interval from X minus 5% of X to X plus 5% of X.

The use of a numerical range in this description is intended unambiguously to include within the scope of the invention all individual integers within the range and all the combinations of upper and lower limit numbers within the broadest scope of the given range.

As used herein, the term “comprising” is to be read as meaning a fixed dose combination of the agents which are stated comprise the composition of the invention, such that the components are mixed together as part of the manufacturing process, forming an essentially homogenous mixture. For the avoidance of doubt, the co-administration of the two agents that comprise the composition of the invention, even if simultaneous, would not constitute a “mixture” as defined herein. However, as noted above, chemical combinations of the components which comprise the mixture (such as compound VI) is envisaged, and constitutes a mixture in accordance with this definition.

As used herein, the term “TGF-beta Production Stimulator” is used to describe an agent that increases cellular production of the cytokine, TGF-beta. Methods to determine whether an agent is a TGF-beta Production Stimulator are well known in the art (see for example U.S. Pat. No. 6,410,587 which is incorporated by reference herein). A suitable test to determine whether (and to what extent) an agent is a TGF-beta Production Stimulator is provided in Example 1, but other equivalent tests could also be used, as described elsewhere.

As used herein, the term “TOE-beta” is used to mean the mammalian TGF-beta1 isoform specifically (unless expressly stated otherwise, or obvious from the context of the usage).

As used herein, the term “Droloxifene” is used to mean the compound (E)-1-(4′-(2-dimethylaminoethoxy)phenyl)-1-(3-hydroxyphenyl)-2-phenylbut-1-ene, shown in formula IV, and encompasses the free base as well as any salt form of the compound, in any hydration state.

As used herein, the term “Clopidogrel” is used to mean the compound Methyl 2-(2-chlorophenyl)-2-(6,7-dihydrothieno[3,2-c]pyridin-5(4H)-yl)acetate, shown in formula V, and encompasses the free base as well as any salt form of the compound, in any hydration state, with the stereocentre in the (S) configuration.

The term ‘CLP*’ is used to mean the compound (Z,1S) 3′-carboxymethylene-4′-mercapto-piperidin-1-yl-(2″-chlorophenyl)acetate, generally accepted to be the active netabolite of clopiodgrel. The other stereocentre in the molecule (at the 4-position) may be in either the (R) or the (S) configuration, because it is not known which of these enantiomers is responsible for the biological activity of metabolized clopidogrel in vivo (see Pereillo et al. (2002) Drug Met. Disposition 30:1288-95).

Unless otherwise defined, all the technical and scientific terms used here have the same meaning as that usually understood by an ordinary specialist in the field to which this invention belongs. Similarly, all the publications, patent applications, all the patents and all other references mentioned here are incorporated by way of reference (where legally permissible).

### FIGURES

FIG. 1 shows the pathways involved in the regulation and activation of TGF-beta. The diagram is based on specific data for TGF-beta1, but very similar pathways operate for TGF-beta2 and TGF-beta3. A TGF-beta Production Stimulator, as defined herein, can act on any of these process (or others not illustrated here) in order to increase the amount of local latent TGF-beta available for one or more of the steps marked ‘activation’.

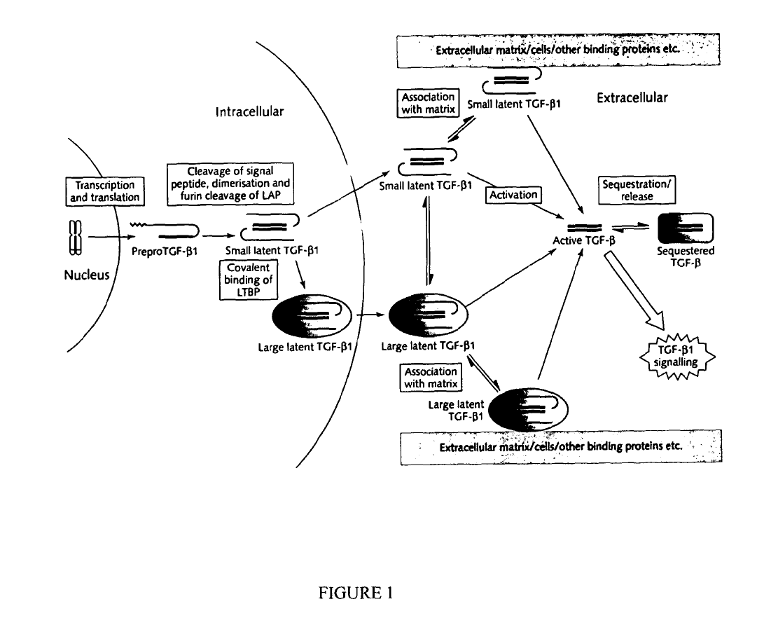

FIG. 2 shows the dose-response curves for the TGF-beta1 dependent inhibition of proliferation of cultures of rat aortic smooth muscle cells in the presence of various concentrations of the structural analogs of tamoxifen used.

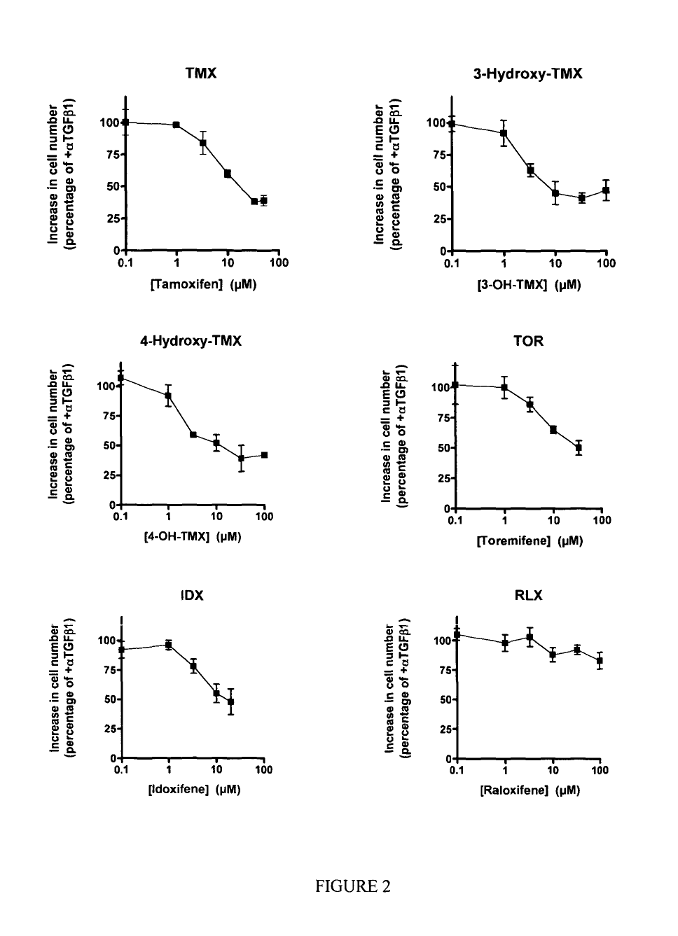

1. Use of a composition, comprising a mixture of droloxifene and clopidogrel, or the pharmaceutically acceptable salts thereof, for the manufacture of a medicament intended to treat or prevent a disorder associated with the loss of normal adult tissue architecture.

2. A pharmaceutical composition comprising a mixture of droloxifene and clopidogrel, or the pharmaceutically acceptable salts thereof, for use as a medicament intended to treat or prevent a disorder associated with the loss of normal adult tissue architecture.

3. A mixture of droloxifene and clopidogrel, or the pharmaceutically acceptable salts thereof, wherein the mixture is essentially homogeneous.

4. A mixture of claim 3 where the pharmaceutically acceptable salt of droloxifene is droloxifene citrate.

5. A mixture of claim 3 where the pharmaceutically acceptable salt of clopidogrel is clopidogrel bisulphate.

6. A mixture of claim 3 where the molar ratio of the two components is between 10:1 and 1:10.

7. A composition, according to claim 2 where the pharmaceutically acceptable salt of droloxifene is droloxifene citrate.

8. Use of a compound of formula (VI) or a pharmaceutically acceptable salt thereof, for the preparation of a medicament intended to treat an inflammatory disorder:

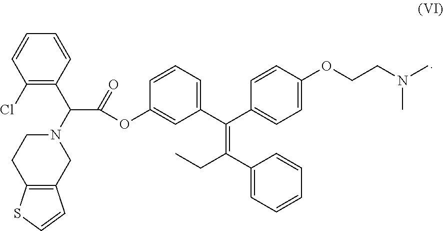 [SMILES: CC/C(=C(/c1ccc(OCCN(C)C)cc1)c1cccc(OC(=O)C(c2ccccc2Cl)N2CCc3sccc3C2)c1)c1ccccc1]

9. Use of a compound of formula (VI′) or a pharmaceutically acceptable salt thereof, for the preparation of a medicament intended to treat an inflammatory disorder:

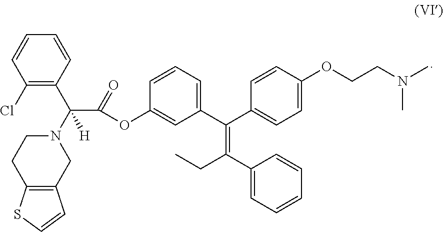 [SMILES: CC/C(=C(/c1ccc(OCCN(C)C)cc1)c1cccc(OC(=O)[C@H](c2ccccc2Cl)N2CCc3sccc3C2)c1)c1ccccc1]

10. A pharmaceutically acceptable composition comprising, as active ingredient, a compound of formula (VI) or a pharmaceutically acceptable salt thereof, and at least one pharmaceutically acceptable excipient and/or carrier:

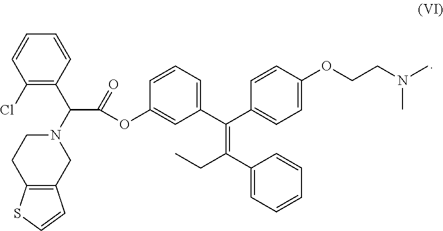 [SMILES: CC/C(=C(/c1ccc(OCCN(C)C)cc1)c1cccc(OC(=O)C(c2ccccc2Cl)N2CCc3sccc3C2)c1)c1ccccc1]

11. A pharmaceutically acceptable composition comprising, as active ingredient, a compound of formula (VI′) or a pharmaceutically acceptable salt thereof, and at least one pharmaceutically acceptable excipient and/or carrier:

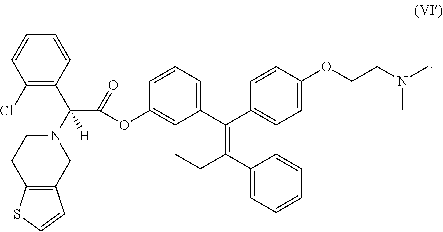 [SMILES: CC/C(=C(/c1ccc(OCCN(C)C)cc1)c1cccc(OC(=O)[C@H](c2ccccc2Cl)N2CCc3sccc3C2)c1)c1ccccc1]

12. A compound of general formula (VI):

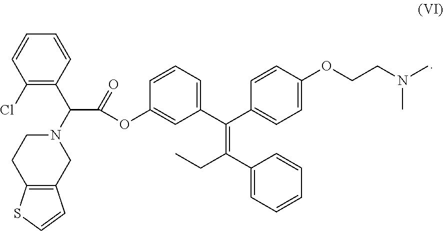

13. A compound of general formula (VI′):

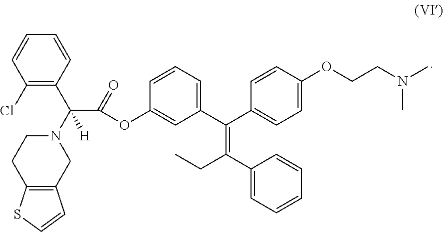

14. A composition, comprising a mixture of droloxifene and clopidogrel, or the pharmaceutically acceptable salts thereof, for the treatment or prevention of a disorder associated with the loss of normal adult tissue architecture.

15. A compound of formula (VI) or (VI′) or a pharmaceutically acceptable salt thereof, for the treatment of an inflammatory disorder: 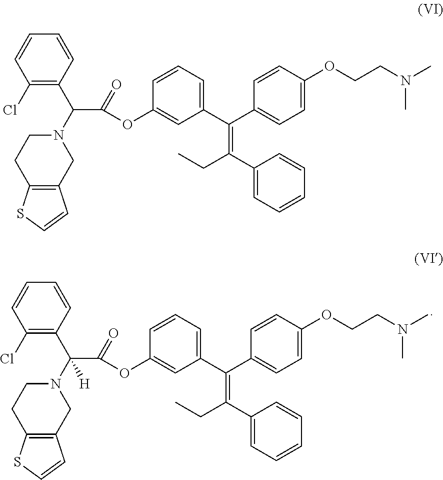 [SMILES: CC/C(=C(/c1ccc(OCCN(C)C)cc1)c1cccc(OC(=O)C(c2ccccc2Cl)N2CCc3sccc3C2)c1)c1ccccc1 ; CC/C(=C(/c1ccc(OCCN(C)C)cc1)c1cccc(OC(=O)[C@H](c2ccccc2Cl)N2CCc3sccc3C2)c1)c1ccccc1]
